(19) United States
(12) Patent Application Publication
(10) Pub. No.: US 2025/0218217 A1
(43) Pub. Date: Jul. 3, 2025

(54) EYE DETECTION APPARATUS AND ELECTRONIC DEVICE

(71) Applicants: Beijing Zitiao Network Technology Co., Ltd., Beijing (CN); Lemon Inc., Grand Cayman (KY)

(72) Inventors: Xin LI, Beijing (CN); Feiya ZHOU, Beijing (CN); Xiaokai LI, Beijing (CN); Bowei ZHANG, Beijing (CN); Runyu LIU, Beijing (CN)

(21) Appl. No.: 18/927,845

(22) Filed: Oct. 25, 2024

(30) Foreign Application Priority Data
Dec. 28, 2023 (CN) 202311843330.2

(51) Int. Cl. G06V 40/19; G06V 10/141; G06V 10/145; G06V 20/64

(52) U.S. Cl. CPC G06V 40/19; G06V 10/141; G06V 10/145; G06V 20/64

(57) ABSTRACT

Embodiments of the disclosure provide an eye detection apparatus and an electronic device. The apparatus includes a transmitting-side assembly and a receiving-side assembly. The transmitting-side assembly includes a light source and a light modulation element, wherein the light modulation element is configured to modulate light emitted by the light source to generate a patterned light beam for forming discrete light spots on an eye. The receiving-side assembly includes a camera configured to acquire an eye image including an image of the discrete light spots to create three-dimensional topographical information of the eye.

### CROSS-REFERENCE

The present application claims priority to Chinese Patent Application No. 202311843330. 2, filed on Dec. 28, 2023, and entitled “EYE DETECTION APPARATUS AND ELECTRONIC DEVICE”, the entirety of which is incorporated herein by reference.

### FIELD

Embodiments of the present disclosure generally relate to the technical field of eye information acquisition, and more particularly, to an eye detection apparatus and an electronic device.

### BACKGROUND

Mixed reality (MR) is a mixture of physical and digital worlds, enabling natural and intuitive three-dimensional (3D) interactions between people, computers, and environments, which are typically implemented based on computer vision, graphics processing, and optical display, among other technologies.

The MR optical display system can transmit digital information, including text, graphics, and video streams and the like, to a user through a complex optical system, so that the user can obtain a more realistic, more immersive, and virtual-reality-integrated visual experience. In actual use, an interpupillary distance (IPD) of the user, a longitudinal direction (also referred to as a Z direction) distance (also referred to as an eye relief, ER) of the eye of the user from the MR optical system, and a transverse direction (also referred to as an XY direction) distance of the eyeball center from the central axis of the optical system may affect the display effect of the MR optical display system. In addition, eye tracking (ET) also needs to obtain relatively accurate gaze point angle and pupil position information, thereby implementing applications such as user intention understanding and analysis, gaze point rendering and eye movement interaction and the like.

Therefore, obtaining accurate eye three-dimensional contour information not only can assist the MR optical display system in eye relief, pupil distance and wearing adjustment, but also can help eye tracking to obtain more accurate position and pose information, thereby realizing more natural and efficient interaction perception.

A conventional eye tracker operates mainly based on the principle of pupil corneal reflection. For example, the conventional eye tracker is configured with one or more cameras for capturing an image of the eye of the user. After obtaining the image, the back-end algorithm may use an image processing algorithm to identify the pupil center and the position of the corneal reflection center on each image, and achieve the determination of the pupil position. However, this conventional solution is only suitable for two-dimensional (2D) information acquisition, enabling the detection of the pupil position in the XY direction, and cannot measure the eye relief in the Z direction, thus failing to generate a fine three-dimensional contour of the eye.

### SUMMARY

In a first aspect of the present disclosure, there is provided an eye detection apparatus, including: a transmitting-side assembly including a light source and a light modulation element, wherein the light modulation element is configured to modulate light emitted by the light source to generate a patterned light beam for forming discrete light spots on an eye; and a receiving-side assembly including a camera configured to acquire an eye image including an image of the discrete light spots to create three-dimensional topographical information of the eye.

In a second aspect of the present disclosure, there is provided an electronic device, including the eye detection apparatus according to the first aspect of the present disclosure.

It should be understood that the content described in this content section is not intended to limit the key features or important features of the embodiments of the present disclosure, nor is it intended to limit the scope of the present disclosure. Other features of the present disclosure will become readily understood from the following description.

### BRIEF DESCRIPTION OF THE DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIGS. 1 to 4 illustrate schematic structural diagrams of an electronic device according to some embodiments of the present disclosure;

FIG. 5 illustrates a schematic structural diagram of a transmitting-side assembly according to some embodiments of the present disclosure;

FIGS. 6 and 7 illustrate schematic diagrams of a field of view of a patterned light beam according to some embodiments of the present disclosure;

FIGS. 8 and 9 illustrate example arrangements of photoelectric detector according to some embodiments of the present disclosure;

FIG. 10 illustrates a schematic structural diagram of a light modulation element according to some embodiments of the present disclosure;

FIG. 11 illustrates a schematic structural diagram of a transmitting-side assembly according to some embodiments of the present disclosure; and

FIGS. 12 and 13 illustrate defects of diffractive optical elements according to some embodiments of the present disclosure.

### DESCRIPTION OF REFERENCE NUMERALS

1 transmitting-side assembly;
10 light source;
101 light;
102 sub-light source;
11 light modulation element;
111 diffractive optical element;
112 grating structure;
113 patterned light beam;
1131 first field of view;
1132 second field of view;
114 discrete light spots;
115 transparent conductive layer;
116 electrode;
12 collimating lens;
2 receiving-side assembly;
20 camera;
30 lens barrel;
31 display module;
32 lens module;
321 semi-transmissive and semi-reflective film;
322 quarter-wave plate;
323 reflective polarizing film;
33 LED;
4 photoelectric detector;
51 fracture portion;
52 missing portion;
80 eye.

### DETAILED DESCRIPTION

Preferred embodiments of the present disclosure will be described in more detail below with reference to the accompanying drawings. While the preferred embodiments of the present disclosure are shown in the drawings, it should be understood that the present disclosure may be implemented in various forms and should not be limited by the embodiments set forth herein. Rather, these embodiments are provided to make this disclosure more thorough and complete, and to fully convey the scope of the present disclosure to those skilled in the art.

As used herein, the term “comprising” and variations thereof represent openness, i.e., “including but not limited to”. Unless specifically stated, the term “or” means “and/or”. The term “based on” means “based at least in part on”. The terms “an example embodiment” and “an embodiment” mean “at least one example embodiment”. The term “another embodiment” means “at least one further embodiment”. The terms “first” “second” and the like may refer to different or identical objects.

As described above, the conventional eye tracker is only suitable for two-dimensional (2D) information acquisition, and cannot measure the eye relief in the Z direction, thus failing to generate a fine three-dimensional contour of the eye. The embodiments of the disclosure provide a solution for acquiring the topographical information of the eye, and can obtain complete three-dimensional information of the eye. The principles of the present disclosure will be described below with reference to the accompanying drawings.

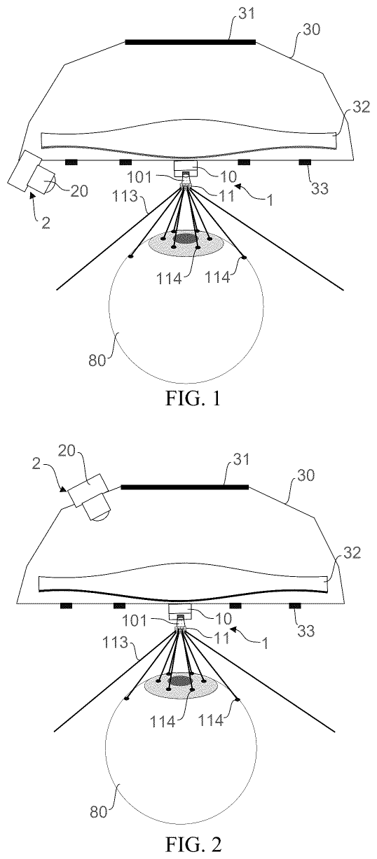

FIGS. 1 to 4 are schematic structural diagrams of an electronic device according to some embodiments of the present disclosure. The solution for acquiring the topographical information of the eye provided by the embodiments of the present disclosure may be implemented in the example electronic devices shown in FIGS. 1 to 4. The electronic devices described herein may be mixed reality (MR) devices or other devices with eye tracking (ET) function. In such devices, the solutions for acquiring the topographical information of the eye may be integrated to obtain three-dimensional topographical information of the eye of the user. It should be understood that the solutions for acquiring the topographical information of the eye according to the embodiments of the present disclosure may also be applied to other types of electronic devices to obtain three-dimensional topographical information of the eye of the user.

As shown in FIG. 1, the electronic device includes a lens barrel 30, a lens module 32 disposed in the lens barrel 30, and a display module 31 disposed on a back side of the lens barrel 30. The display module 31 may display digital information, such as text, graphics, and video streams, which may be transmitted to the user via the lens module 32. In some embodiments, a plurality of light emitting diodes (LEDs) 33 may also be disposed on the front side of the lens barrel 30, so as to assist in realizing the eye tracking function.

As shown in FIGS. 1 to 4, the electronic device integrates an eye detection apparatus including a transmitting-side assembly 1 and a receiving-side assembly 2. The transmitting-side assembly 1 includes a light source 10 and a light modulation element 11. The light source 10 may generate light 101 having a certain wavelength or a certain band of wavelength. The light modulation element 11 may modulate the light 101 emitted by the light source 10 to generate a patterned light beam 113 for forming discrete light spots 114 on an eye 80. The receiving-side assembly 2 includes a camera 20, and the camera 20 may acquire an eye image including an image of the discrete light spots 114, so as to create three-dimensional topographical information of the eye.

It should be understood that in FIGS. 1 to 4, only a few of the light beams of the patterned light beam 113 and several light spots of the discrete light spots 114 are shown as examples to illustrate the operating principle of the transmitting-side assembly 1. Indeed, according to design requirements, the transmitting-side assembly 1 may generate any suitable patterned light beam 11 to form any suitable number of discrete light spots 114 on the eye 80.

In an embodiment, as shown in FIG. 1, both the transmitting-side assembly 1 and the receiving-side assembly 2 are disposed outside the lens barrel 30. With this arrangement, the patterned light beam 113 emitted by the transmitting-side assembly 1 can irradiate directly onto the eye 80, thereby forming discrete light spots 114 on the eye 80. An eye image including a reflected image of the discrete light spots 114 may be acquired by the camera 20 to create three-dimensional topographical information of the eye 80.

In an embodiment of the present disclosure, the three-dimensional reconstruction of the eye topography may be performed based on the eye image including the reflected image of the discrete light spots 114 using any suitable method, for example, it may be based on a three-dimensional ranging method, a feature matching method, or the like.

In an embodiment, as shown in FIG. 2, the transmitting-side assembly 1 is disposed outside the lens barrel 30, and the receiving-side assembly 2 is disposed in the lens barrel 30. With this arrangement, the patterned light beam 113 emitted by the transmitting-side assembly 1 can irradiate directly onto the eye 80, thereby forming discrete light spots 114 on the eye 80. In turn, an eye image including a reflected image of the discrete light spots 114 may be acquired by the camera 20 via the lens module 32 to create three-dimensional topographical information of the eye 80.

In an embodiment, as shown in FIG. 3, the transmitting-side assembly 1 is disposed in the lens barrel 30, and the receiving-side assembly 2 is disposed outside the lens barrel 30. With this arrangement, the patterned light beam 113 emitted by the transmitting-side assembly 1 can pass through the lens module 32 to illuminate the eye 80, thereby forming discrete light spots 114 on the eye 80. In turn, an eye image including a reflected image of the discrete light spots 114 may be acquired by the camera 20 to create three-dimensional topographical information of the eye 80.

In an embodiment, as shown in FIG. 4, both the transmitting-side assembly 1 and the receiving-side assembly 2 are disposed in the lens barrel 30. With this arrangement, the patterned light beam 113 emitted by the transmitting-side assembly 1 can pass through the lens module 32 to illuminate the eye 80, thereby forming the discrete light spots 114 on the eye 80. In turn, an eye image including the reflected image of the discrete light spots 114 may be acquired by the camera 20 via the lens module 32 to create three-dimensional topographical information of the eye 80.

In some embodiments, the wavelength of light 101 emitted by light source 10 may be located in the visible band. In other embodiments, the wavelength of the light 101 emitted by the light source 10 may be in the near infrared band. In other embodiments, the wavelength of the light 101 emitted by the light source 10 may be located in other wave bands, as long as it is able to form the discrete light spots 114 on the eye 80 whose image can be acquired by the camera 20. The light 101 emitted by the light source 10 may have a particular morphology of image, such as a sinusoidal stripe, a dot, a single line, multi-lines, regular speckles, random speckles, or the like.

In some embodiments, the light source 10 may include a vertical-cavity surface-emitting laser (VCSEL), a laser diode (LD), or a light-emitting diode (LED). In other embodiments, the light source 10 may also be of other types, and these implementations also fall within the scope of the present disclosure.

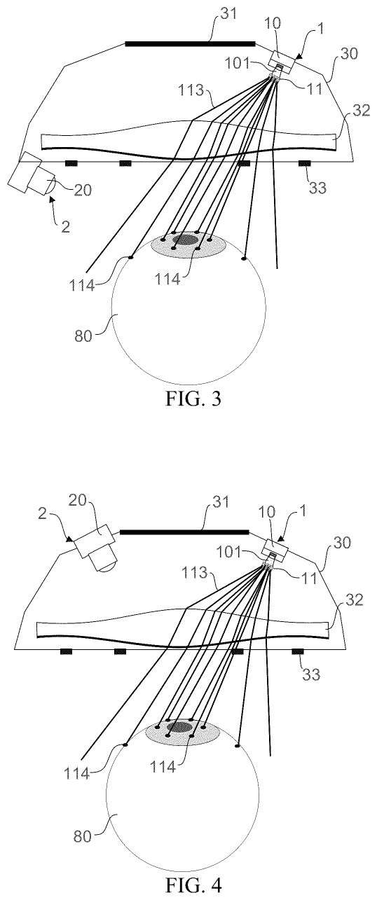

In some embodiments, the light source 10 may be a surface-emitting light source or an edge-emitting light source. In other embodiments, the light source 10 may include a plurality of sub-light sources 102, as shown in FIG. 5. FIG. 5 illustrates a schematic structural diagram of a transmitting-side assembly according to some embodiments of the present disclosure. Only a few sub-light sources 102 are shown in FIG. 5 as examples, and in actual embodiments, the number of sub-light sources 102 may reach hundreds, thousands, or even more. The sub-light sources 102 may be arranged in any suitable form. For example, the sub-light sources 102 may be regularly arranged or irregularly arranged. The sub-light sources 102 may be arranged in one or two dimensions.

In some embodiments, the light 101 emitted by the light source 10 may be circularly polarized or linearly polarized.

In some embodiments, as shown in FIGS. 1 to 4, the light emitting surface of the light source 10 may be spaced apart from the light modulation element 11 by a certain distance, that is, there may be a gap therebetween. The light 101 emitted by the light source 10 may irradiate onto the light modulation element 11 via the gap. In some embodiments, as shown in FIG. 5, the transmitting-side assembly 1 further includes a collimating lens 12. The collimating lens 12 is disposed between the light source 10 and the light modulation element 11 for collimating the light 101 emitted by the light source 10 to reduce the beam emission angle. The collimating lens 12 may direct the collimated light 101 onto the light modulation element 11.

In some embodiments, the light modulation element 11 may be directly integrated on the light emitting surface of the light source 10, and directly modulate the light 101 emitted by the light source 10. In this way, the volume and cost of the transmitting-side assembly 1 may be reduced.

In one embodiment, as shown in FIG. 5, the light modulation element 11 includes a diffractive optical element 111. Diffractive optical element 111 may split the light 101 emitted by light source 10 to generate patterned light beam 113.

In some embodiments, as shown in FIG. 5, the incident surface of the diffractive optical element 111 is provided with a series of grating structures 112 having a predetermined period and depth. The grating structures 112 may control the diffraction direction and the diffraction intensity of the light beam incident thereon to achieve a specific diffraction pattern topography. In some embodiments, the grating structures 112 are one-dimensional grating structures or two-dimensional grating structures. The one-dimensional grating structures have a periodicity in one direction. The two-dimensional grating structures have a periodicity in two directions orthogonal to each other.

In some embodiments, the diffractive optical element 111 may be made of resin, polycarbonate (PC), glass, or liquid crystal. It should be understood that the diffractive optical element 111 may be made of any suitable material as long as it is able to diffract the light 101 emitted by the light source 10 to generate the patterned light beam 113.

In some embodiments, the diffractive optical element 103 is a diffractive element combination, which may include one layer of diffractive structure, two layers of diffractive structures, or more layers of diffractive structures. Each layer of the diffractive structures may diffract the light respectively to obtain a desired patterned light beam 113.

In some embodiments, the light modulation element 11 may include a metalens instead of the diffractive optical element 111. With the metalens, the light 101 emitted by the light source 10 can also be modulated to generate a patterned light beam 113 for forming discrete light spots 114 on the eye 80.

In some embodiments, as shown in FIGS. 1 to 4, the receiving-side assembly 2 may multiplex a camera module configured to implement an eye tracking function of the electronic device. With this arrangement, the structure of the electronic device can be simplified and the cost of the electronic device can be reduced. In some other embodiments, the receiving-side assembly 2 may be a camera module independent of the eye tracking function of the electronic device.

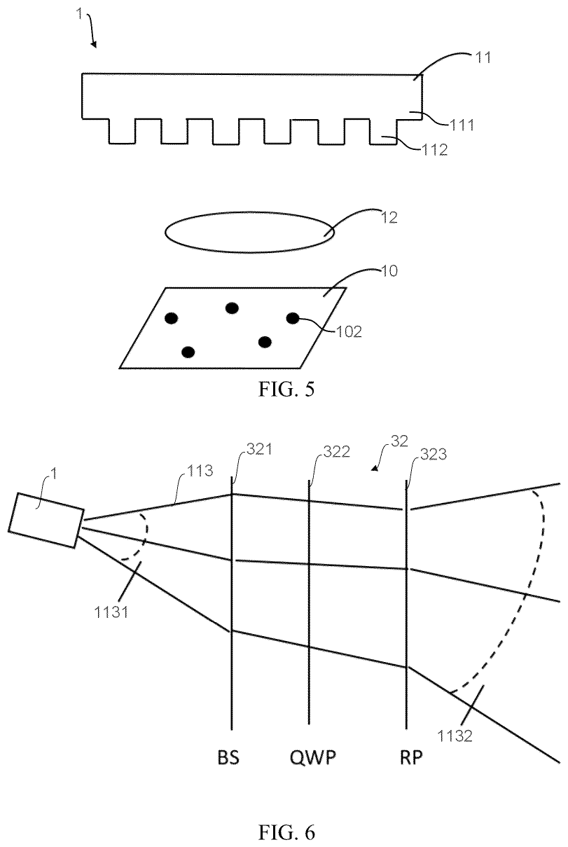

In some embodiments, the transmitting-side assembly 1 is disposed in the lens barrel 30, which can optimize the structural parameters (e.g., radius, surface type, etc.) of the lens module 32, so that the field of view of the patterned light beam 113 emitted by the transmitting-side assembly 1 can be further increased after being refracted by the lens module 32. FIGS. 6 and 7 show schematic diagrams of the field of views of patterned light beams 113 according to some embodiments of the present disclosure. As shown in FIG. 6, the patterned beam 113 has a first field of view 1131 before entering the lens module 32, and a second field of view 1132 after passing through the lens module 32. The first field of view 1131 of the patterned light beam 113 before reaching the lens module 32 is smaller than the second field of view 1132 of the patterned light beam 113 after passing through the lens module 32. In this way, the projected illumination area of the patterned light beam 113 is expanded.

In some embodiments, the lens module 32 may include at least one of a Fresnel lens, a spherical lens group, a non-spherical lens group, or a folded mirror group. It should be understood that the lens module 32 may include any suitable type of lens, which is not limited in the embodiments of the present disclosure.

In an embodiment, as shown in FIG. 6, in a case where the folded mirror group is used, the folded mirror group may include a film material such as a semi-transmissive and semi-reflective film (Beam Splitter (BS)) 321, a reflective polarizing film (RP) 323, and a quarter-wave plate (QWP) 322 and the like.

In some embodiments, in the case where the foldback mirror group is used, the properties of the film materials such as the semi-transmissive and semi-reflective film 321, the reflective polarizing film 323, and the quarter-wave plate 322 and the like, such as the transmittance, the reflectivity, the loss, the phase retardation and the like, may be optimized, so that a part of the patterned light beam 113 emitted by the transmitting-side assembly 1 directly penetrates through the lens module 32 to irradiate the eye 80 (as indicated by the solid line in FIG. 7), and another part of the patterned light beam 113 irradiates the eye 80 after undergoing a foldback propagation in the lens module 32 (as indicated by the dotted line in FIG. 7). In this way, the number of light spots 114 near the eye 80 can be significantly increased, thereby more accurately determining the three-dimensional topography of the eye 80.

Since the light source 10, such as a laser source and the like, is used as the illumination element, in order to avoid the safety risk that would arise due to excessively high power of the illumination element and too long irradiation pulse width time, a protection mechanism may be provided.

In some embodiments, the transmitting-side assembly 1 is disposed in the lens barrel 30, and the electronic device may further include a photoelectric detector 4. The photodetector 4 is disposed in the lens barrel 30 and may detect an optical signal reflected by the lens module 32. When the intensity of the optical signal detected by the photoelectric detector 4 is greater than a predetermined threshold, it may be determined that the intensity of the patterned light beam 113 emitted by the transmitting-side assembly 1 is too large, and there is a safety risk. Therefore, the operating parameters of the transmitting-side assembly 1, such as the current, voltage, duty cycle, etc. in the light source 10, may be dynamically adjusted, thereby reducing the output power of the light source 10, and even in some cases, the light source 10 can be turned off directly. In this way, the eye safety of the user can be ensured.

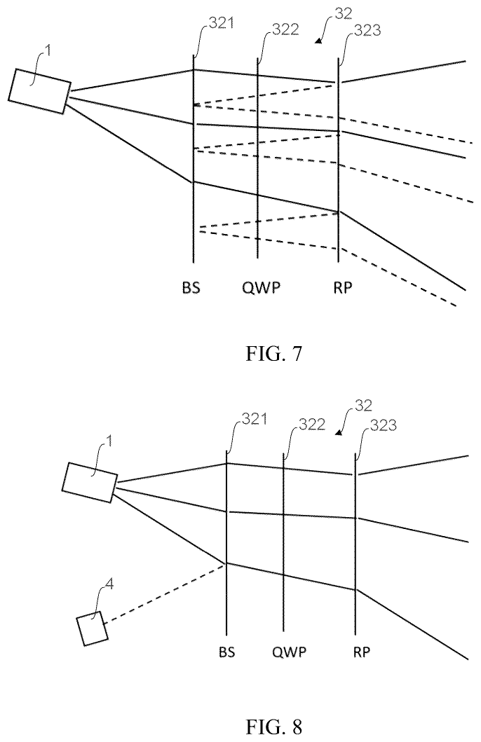

FIGS. 8 and 9 illustrate example arrangements of photoelectric detector 4 according to some embodiments of the present disclosure. In some embodiments, as shown in FIG. 8, the photoelectric detector 4 is disposed on an inner side of the lens module 32, that is, a side of close to the transmitting-side assembly 1. In some embodiments, as shown in FIG. 9, the photoelectric detector 4 is disposed between different units of different lens modules 32. For example, the photodetector 4 may be disposed between the semi-transmissive and semi-reflective film 321 and the quarter-wave plate 322, or between the quarter-wave plate 322 and the reflective polarizing film 323.

In some embodiments, the transmitting-side assembly 1 includes a power detection element for detecting the output power of the light source 10. As an example, the light source 10 may be provided with a photodiode for detecting the output power of the light source 10, avoiding excessively high power of the light source 10, and further ensuring the safety of the human eye.

In some embodiments, the distribution of the discrete light spots 114 may be detected by utilizing the camera 20, and if the energy of the emitted light spot is too concentrated or excessively high, the light source 10 may be turned off.

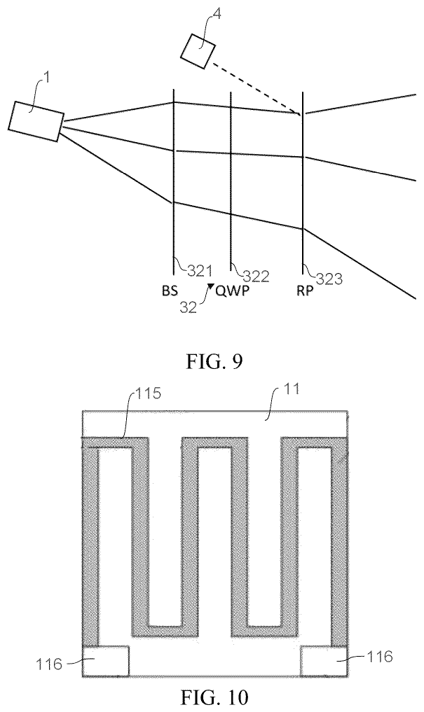

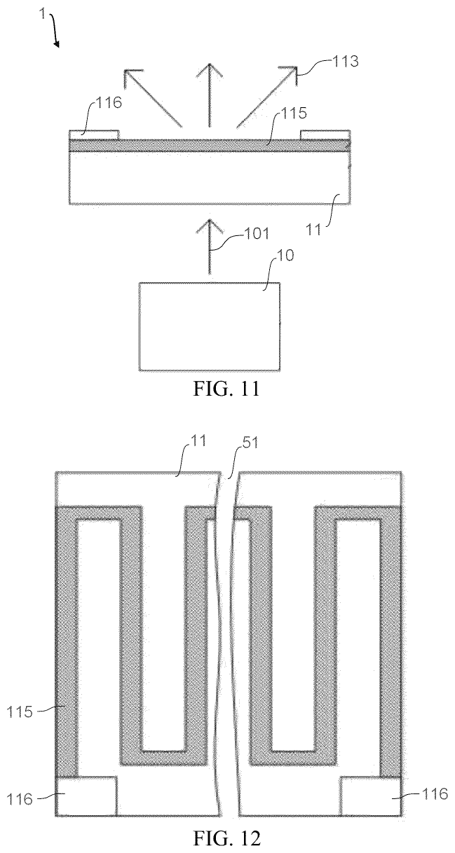

In some embodiments, a transparent conductive material may be formed on a side of the light modulation element 11 facing away from the light source 10 to detect a device state of the light modulation element 11, as shown in FIGS. 10 and 11. FIG. 10 illustrates a schematic structural diagram of a light modulation element 11 according to some embodiments of the present disclosure, and FIG. 11 shows a schematic structural diagram of a transmitting-side assembly 1 according to some embodiments of the present disclosure.

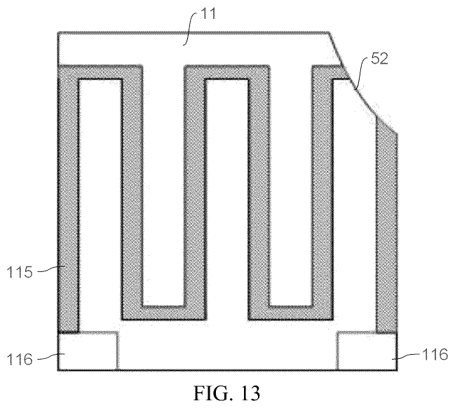

In some embodiments, as shown in FIGS. 10 and 11, a side of the light modulation element 11 facing away from the light source 10 is provided with a transparent conductive layer 115 and a pair of electrodes 116. The light modulation element 1 1 is generally of a rectangular shape. One of the pair of electrodes 116 is disposed at one corner of the light modulation element 11 and electrically connected to an end of the transparent conductive layer 115. The other of the pair of electrodes 116 is disposed at another corner of the light modulation element 11 and is electrically connected to the other end of the transparent conductive layer 115. The two corners of the light modulation element 11 may be two corners on a diagonal of the light modulation element 11, or may be two corners on a side edge. The transparent conductive layer 115 extends along a foldback path between the paired electrodes 116, and is generally in a bending shape. The transparent conductive layer 115 may be connected to the monitoring circuit through the pair of electrodes 116. When the light modulation element 11 is complete and free of any damage, the electrical signal in the monitoring circuit remains stable, indicating that the light modulation element 11 is in a normal operating state. When the light modulation element 11 is damaged or broken, the electrical signal in the monitoring circuit may be abnormal, thereby indicating that the light modulation element 11 is in an abnormal operating state. FIGS. 12 and 13 illustrate defects of diffractive optical elements 11 according to some embodiments of the present disclosure. As shown in FIG. 12, the light modulation element 11 has a fracture portion 51 thereon. As shown in FIG. 13, the light modulation element 11 has a missing portion 52 thereon. The fracture portion 51 and the missing portion 52 cause the transparent conductive layer 115 to be disconnected. In this case, the state of the light modulation element 11 can be determined in time according to the change of the electrical signal, and the light source 10 can be controlled to be turned on or turned off to avoid a safety risk.

In some embodiments, the transparent conductive layer 115 includes at least one of indium tin oxide (ITO) or graphene. It should be understood that other types of transparent conductive materials are possible, which is not limited in the scope of the present disclosure.

It should be understood that the bent transparent conductive layer 115 is merely an example. In other embodiments, the transparent conductive layer 115 is of a linear shape or a ring shape or any other available shape. As an example, the pair of electrodes 116 may be disposed at two corners on a diagonal of the light modulation element 11, and the transparent conductive layer 115 extends along a straight path between the pair of electrodes 116. In this case, the transparent conductive layer 115 is generally linear in shape. As another example, the pair of electrodes 116 may be disposed at two corners on a diagonal of the light modulation element 11 or at two corners on a side edge of the light modulation element 11, and the transparent conductive layer 115 may start at one electrode 116 and wrap inwardly, and then wrap outwardly after wrapping around to a center point, and finally reaching the other electrode 116. In this case, the transparent conductive layer 115 is generally in a wrap-around shape.

According to the embodiments of the present disclosure, it is able to obtain the complete three-dimensional information of the eye can be obtained, which breaks through the limitation that the traditional eye tracking solution can only calculate the two-dimensional plane topographical information, and expands more possibilities for the application of the MR device and the eye tracker. In addition, according to an embodiment of the present disclosure, imaging ghost images can be reduced. Due to the collimation characteristics of the VCSEL and the diffractive optical element, most of the signals received on the receiving-side assembly are signals generated by eye scattering, which have good anti-interference characteristics.

Embodiments of the present disclosure are also embodied in the following examples.

Example 1. An eye detection apparatus, including: a transmitting-side assembly including a light source and a light modulation element, wherein the light modulation element is configured to modulate light emitted by the light source to generate a patterned light beam for forming discrete light spots on an eye; and a receiving-side assembly including a camera configured to acquire an eye image including an image of the discrete light spots to create three-dimensional topographical information of the eye.

Example 2. The eye detection apparatus of example 1, wherein the light source includes at least one of: a surface-emitting light source; an edge-emitting light source; or a plurality of sub-light sources.

Example 3. The eye detection apparatus of example 1, wherein the transmitting-side assembly includes a power detection element configured to detect an output power of the light source.

Example 4.: The eye detection apparatus of example 1, wherein the light modulation element includes a diffractive optical element configured to split the light emitted by the light source to generate the patterned beam.

Example 5. The eye detection apparatus of example 4, wherein the diffractive optical element includes a grating structure having a predetermined period and depth.

Example 6. The eye detection apparatus of example 5, wherein the grating structure is a one-dimensional grating structure or a two-dimensional grating structure.

Example 7. The eye detection apparatus of Example 4, wherein the diffractive optical element includes one or more layers of diffractive structures.

Example 8. The eye detection apparatus of example 1, wherein the light modulation element is integrated on a light-emitting surface of the light source.

Example 9. The eye detection apparatus of example 1, wherein the light modulation element includes a metalens.

Example 10. The eye detection apparatus of example 1, wherein the transmitting-side assembly further includes: a collimating lens disposed between the light source and the light modulation element and configured to collimate light emitted by the light source and direct the collimated light onto the light modulation element.

Example 11. The eye detection apparatus of example 1, wherein a side of the light modulation element facing away from the light source is provided with a transparent conductive layer and a pair of electrodes, one of the pair of electrodes is connected to an end of the transparent conductive layer, and the other of the pair of electrodes is connected to the other end of the transparent conductive layer.

Example 12. The eye detection apparatus of example 11, wherein the transparent conductive layer includes at least one of indium tin oxide or graphene.

Example 13. The eye detection apparatus of example 11, wherein the transparent conductive layer is of a rectangular shape, the pair of electrodes (116) are disposed at two corners of the transparent conductive layer (115), and the transparent conductive layer (115) extends along a foldback path, a straight path, or a wrap-around path between the pair of electrodes (116).

Example 14. An electronic device, including the eye detection apparatus of any one of examples 1 to 13.

Example 15. The electronic device of example 14, wherein the electronic device includes a lens barrel and a lens module disposed in the lens barrel, wherein the transmitting-side assembly is disposed in the lens barrel or outside the lens barrel, and the receiving-side assembly is disposed in the lens barrel or outside the lens barrel.

Example 16. The electronic device of example 15, wherein the transmitting-side assembly is disposed in the lens barrel, the electronic device further includes a photoelectric detector disposed in the lens barrel and configured to detect an optical signal reflected by the lens module, wherein when the intensity of the optical signal is greater than a predetermined threshold, an output power of the light source is reduced or the light source is turned off.

Example 17. The electronic device of example 16, wherein the photoelectric detector is disposed on a side of the lens module close to the transmitting-side assembly or between different units of different lens modules.

Example 18. The electronic device of example 15, wherein the transmitting-side assembly is disposed in the lens barrel, and a first field of view of the patterned light beam emitted by the transmitting-side assembly before reaching the lens module is less than a second field of view of the patterned light beam after passing through the lens module.

Example 19. The electronic device according to example 15, wherein the transmitting-side assembly is disposed in the lens barrel, a part of the patterned light beam emitted by the emission-side assembly directly penetrates through the lens module to irradiate the eye, and another part of the patterned light beam irradiates the eye after undergoing a foldback propagation in the lens module.

Various embodiments of the present disclosure have been described above, which are illustrative, not exhaustive, and are not limited to the embodiments disclosed. Many modifications and variations will be apparent to those of ordinary skill in the art without departing from the scope and spirit of the illustrated embodiments. The selection of the terms used herein is intended to best explain the principles of the embodiments, practical applications, or technical improvements in the marketplace, or to enable others of ordinary skill in the art to understand the embodiments disclosed herein.

I/we claim:

1. An eye detection apparatus, comprising:
a transmitting-side assembly comprising a light source and a light modulation element, wherein the light modulation element is configured to modulate light emitted by the light source to generate a patterned light beam for forming discrete light spots on an eye; and
a receiving-side assembly comprising a camera configured to acquire an eye image comprising an image of the discrete light spots to create three-dimensional topographical information of the eye.

2. The eye detection apparatus of claim 1, wherein the light source comprises at least one of:
a surface-emitting light source;
an edge-emitting light source; or
a plurality of sub-light sources.

3. The eye detection apparatus of claim 1, wherein the transmitting-side assembly comprises a power detection element configured to detect an output power of the light source.

4. The eye detection apparatus of claim 1, wherein the light modulation element comprises a diffractive optical element configured to split the light emitted by the light source to generate the patterned light beam.

5. The eye detection apparatus of claim 4, wherein the diffractive optical element comprises a grating structure having a predetermined period and depth.

6. The eye detection apparatus of claim 5, wherein the grating structure is a one-dimensional grating structure or a two-dimensional grating structure.

7. The eye detection apparatus of claim 4, wherein the diffractive optical element comprises one or more layers of diffractive structures.

8. The eye detection apparatus of claim 1, wherein the light modulation element is integrated on a light-emitting surface of the light source.

9. The eye detection apparatus of claim 1, wherein the light modulation element comprises a metalens.

10. The eye detection apparatus of claim 1, wherein the transmitting-side assembly further comprises:
a collimating lens disposed between the light source and the light modulation element and configured to collimate the light emitted by the light source and direct the collimated light onto the light modulation element.

11. The eye detection apparatus of claim 1, wherein a side of the light modulation element facing away from the light source is provided with a transparent conductive layer and a pair of electrodes, one of the pair of electrodes is connected to an end of the transparent conductive layer, and the other of the pair of electrodes is connected to the other end of the transparent conductive layer.

12. The eye detection apparatus of claim 11, wherein the transparent conductive layer comprises at least one of indium tin oxide or graphene.

13. The eye detection apparatus of claim 11, wherein the transparent conductive layer is of a rectangular shape, the pair of electrodes are disposed at two corners of the transparent conductive layer, and the transparent conductive layer extends along a foldback path, a straight path or a wrap-around path between the pair of electrodes.

14. An electronic device, comprising the eye detection apparatus of any one of claims 1-13.

15. The electronic device of claim 14, wherein the electronic device comprises a lens barrel and a lens module disposed in the lens barrel, wherein the transmitting-side assembly is disposed in the lens barrel or outside the lens barrel, and the receiving-side assembly is disposed in the lens barrel or outside the lens barrel.

16. The electronic device of claim 15, wherein the transmitting-side assembly is disposed in the lens barrel, the electronic device further comprises a photoelectric detector disposed in the lens barrel and configured to detect an optical signal reflected by the lens module, wherein when the intensity of the optical signal is greater than a predetermined threshold, an output power of the light source is reduced or the light source is turned off.

17. The electronic device of claim 16, wherein the photoelectric detector is disposed on a side of the lens module close to the transmitting-side assembly or between different units of different lens modules.

18. The electronic device of claim 15, wherein the transmitting-side assembly is disposed in the lens barrel, and a first field of view of the patterned light beam emitted by the transmitting-side assembly before reaching the lens module is smaller than a second field of view of the patterned light beam after passing through the lens module.

19. The electronic device of claim 15, wherein the transmitting-side assembly is disposed in the lens barrel, a part of the patterned light beam emitted by the transmitting-side assembly directly penetrates through the lens module to irradiate the eye, and another part of the patterned light beam irradiates the eye after undergoing a foldback propagation in the lens module.

20. The electronic device of claim 15, wherein the lens module comprises at least one of a Fresnel lens, a spherical lens group, a non-spherical lens group, or a folded mirror group.
